(19) United States
(12) Patent Application Publication
(10) Pub. No.: US 2014/0284838 A1
(43) Pub. Date: Sep. 25, 2014

(54) APPARATUS AND METHOD FOR EXTRUDING A MEDICAL INSTRUMENT

(71) Applicant: MaRVis Medical GmbH, Frechen (DE)

(72) Inventors: Joachim Georg Pfeffer, Aachen (DE); Martin Schwarz, Oberhausen (DE)

(21) Appl. No.: 14/358,039

(22) PCT Filed: Nov. 16, 2012
(86) PCT No.: PCT/EP2012/004780

§ 371 Date: May 13, 2014

(30) Foreign Application Priority Data
Nov. 16, 2011 (DE) 102011118719.0

(51) Int. Cl. A61M 25/00

(52) U.S. Cl. CPC A61M 25/0009
USPC 264/172.15; 425/505

(57) ABSTRACT

According to the invention, an apparatus is provided for extruding a medical instrument which can be inserted into a human or animal body. The apparatus comprises a device for supplying rod-shaped bodies, an extrusion device comprising a housing, said housing having a surrounding side wall which, at the frontward end as seen in the manufacturing direction, is provided with a nozzle wall comprising a discharge nozzle and, at the rearward end as seen in the manufacturing direction, is provided with a spindle sleeve. The space in the housing between the spindle sleeve, the side wall and the discharge nozzle delimits an extrusion space and the housing is provided with a polymer supply device in the area of the extrusion space. Further, a cannula device is provided which extends in the manufacturing direction and is designed to insert at least one rod-shaped body from the device for supplying rod-shaped bodies into the extrusion space in a predetermined spatial arrangement, which comprises at least one tubular cannula having a rearward supply end as seen in the manufacturing direction and a frontward discharge end as seen in the manufacturing direction, the cannula device being arranged approximately in straight alignment with respect to the discharge nozzle and extending through the spindle sleeve such that its discharge end situated in the manufacturing direction terminates at a distance from the discharge nozzle in the extrusion space.

The present invention relates to an apparatus for extruding a medical instrument which can be inserted into a human or animal body.

Apparatus for extruding cables are known. In the process of extruding cables, a wire is supplied to a spindle sleeve in an extrusion device, a polymer for sheathing the cable being applied to the wire in the extrusion device. In this way, a wire is produced which is sheathed by a plastic material.

Further, bell wires and telephone wires are known in electrical engineering. In their production, a single wire is sheathed with plastic first. For manufacturing multi-wired cables, several of those wires coming from a material loom are joined and then passed through a braiding machine where they are sheathed with a thread braid. These wire bunches will then be inserted into an extrusion device. Here, the wire bunch is directly inserted into the spindle sleeve and then sheathed with plastic.

Document WO 2007/000148 A2 discloses a rod-shaped body which serves for forming medical instruments such as catheters or guide wires for catheters. Said rod-shaped body consists of one or more filaments and a non-ferromagnetic matrix material which encloses the filaments. The matrix material is provided with a doping of particles which create artifacts under magnetic resonance tomography.

Document WO 2009/141165 describes a medical instrument which can be inserted into a human or animal body and comprises an instrument body. The instrument body comprises at least one rod-shaped body which has a poor electrical conductivity and is formed from a matrix material and non-metallic filaments. Said medical instrument is distinguished in that the rod-shaped body is doped with an X-ray marker, and the medical instrument comprises an MR marker.

By providing an X-ray marker as well as an MR marker, the medical instrument is visible both in X-ray examinations and under magnetic resonance tomography. The introduction of the X-ray marker in the medical instrument can be realized in an easy way by using a correspondingly doped rod-shaped body. Such rod-shaped bodies can be manufactured with different dopings at low cost as mass products and with an exact dosage of the marker particles. When manufacturing a medical instrument, the visualization of the medical instrument in X-ray examinations can be ensured by use of the corresponding rod-shaped body containing an X-ray marker.

Said medical instrument can be designed for inserting it into a human or animal body and it comprises an instrument body with a surface which may get into contact with the human or animal body. The surface area of the instrument body may be provided with immobilized MR markers, in particular those containing gadolinium.

Such markers interact with the protons in the water or fat molecule and result in a faster relaxation of the protons neighboring the marker after their orientation induced by the applied magnetic field. The reduction of the relaxation time which is caused by the marking results in strong MRT signals, causing a correspondingly high contrast in the images produced thereby.

Using an immobilized MR marker at the surface of the instrument body in combination with at least one rod-shaped body doped with a marker allows to combine in an easy way the high contrast of a superficially arranged MR marker in magnetic resonance tomography and the versatile range of application of any markers which are distributed in the material of the medical instrument. The markers may be designed for X-ray as well as for magnetic resonance examinations. The medical instrument preferably comprises several rod-shaped bodies which may be doped in different ways.

Medical instruments which have their surface provided with active MR markers possess a very flexible field of application with respect to the sequences which are used in a magnetic resonance tomography examination and are also uniformly visible in magnetic resonance tomography examinations with different sequences.

The superficially arranged MR markers comprise an element or a combination of elements or a compound of an element from the group consisting of gadolinium, cerium, praseodymium, neodymium, promethium, samarium, europium, terbium, dysprosium, holmium, erbium, thulium, ytterbium and lutetium. These elements can be bound as ions in a complex. However, they may also be present in the form of salts or alloys.

It is particularly preferred to use gadolinium as an MR marker. It is immobilized preferably by means of a complex, in particular a chelate complex.

The complexes may either be bound in covalent manner on the surface of the instrument body or embedded in a swellable coating formed on the surface of the instrument body.

The European Patent Application which carries the serial number EP 11 000 937 and is not published yet, discloses a medical instrument comprising a modified cladding polymer and a coating with paramagnetic ions such as gadolinium. The modified cladding polymer comprises chemically active, free functional groups and a coating which is bound to the free functional groups in covalent manner and contains the paramagnetic ions.

For the purpose of sheathing electric cables, extrusion devices are known (e.g.: EP 2 367 177 A1, EP 0 409 011 A1) which supply the individual wires of the cable to an extrusion space via one spindle sleeve.

A similar apparatus is known from EP 1 757 428 A1 for sheathing a catheter or from U.S. Pat. No. 5,451,355 for sheathing a tube braided from fibers.

Document WO 02/20898 A2 discloses a similar extrusion device in which the spindle sleeve comprises a cavity filled with a polymer melt.

Document DE 10 2008 035 573 A1 shows an extrusion tool with which cords or wires are first sheathed with a first polymer and then with a second polymer, wherein these steps are carried out in a single tool.

It is the object of the present invention to provide an apparatus and a method for extruding medical instruments for insertion into a human or animal body, which allows during its manufacture to fulfill specific mechanical characteristics and features regarding the visualization. In particular, it shall be possible to arrange individual rod-shaped bodies in a predetermined position in the medical instrument.

In order to achieve this object, the invention comprises the features as set forth in claims 1, 2 and 13. Advantageous configurations are indicated in the respective sub-claims.

According to the invention, an apparatus is provided for extruding a medical instrument which can be inserted into a human or animal body. The apparatus comprises a device for supplying rod-shaped bodies, an extrusion device comprising a housing, said housing having a surrounding side wall which, at the frontward end as seen in the manufacturing direction, is provided with a nozzle wall comprising a discharge nozzle and, at the rearward end as seen in the manufacturing direction, is provided with a spindle sleeve. The space in the housing between the spindle sleeve, the side wall and the discharge nozzle delimits an extrusion space and the housing is provided with a polymer supply device in the area of the extrusion space. Further, a cannula device is provided which extends in the manufacturing direction and is designed to insert at least one rod-shaped body from the device for supplying rod-shaped bodies into the extrusion space in a predetermined spatial arrangement, which comprises at least one tubular cannula having a rearward supply end as seen in the manufacturing direction and a frontward discharge end as seen in the manufacturing direction, the cannula device being arranged approximately in straight alignment with respect to the discharge nozzle and extending through the spindle sleeve such that its discharge end situated in the manufacturing direction terminates at a distance from the discharge nozzle in the extrusion space.

A cannula device is a device comprising one or more cannulas. A cannula is a thin, straight tube with an essentially constant cross-section. A cannula may have an insertion opening expanded like a funnel, in particular at its inlet opening.

The term “approximately in straight alignment” is to be understood in the context of the present invention in the sense that one or more cannulas are arranged parallel to the manufacturing direction. This does not mean that a cannula is in exact alignment with the center of the discharge nozzle, but that it is arranged parallel to the centerline of the discharge nozzle.

The polymer supply device may be designed for supplying polymers as they are disclosed as a coating in the still unpublished European Patent Application with the serial number EP 11 000 937 to which reference is made hereby in its entirety.

Regarding the medical instrument, reference is made to WO 2009/141165 which has been described above and to which reference is made hereby in its entirety. In the context of the present invention, the term “medical instrument” pertains to guide wires and catheters, for instance. Such medical instruments have a diameter from 0.1 mm up to 5.0 mm. Guide wires have a diameter especially from 0.1 mm to 1.0 mm, preferably from 0.2 to 0.9 mm, and catheters have an outer diameter in particular from 2 FR (French) to 12 FR or from 0.7 mm to 4.0 mm and preferably from 3 FR to 5 FR or 1 mm to 1.7 mm.

The rod-shaped body may be formed from a matrix material which encloses non-metallic filaments and the particles of a respective marker. The matrix material is preferably a plastic material such as epoxy resin, PEEK, PEBAX, PE, PP, PU, silicone, polylactic acid polymers. The filaments are glass fibers, ceramic fibers, Dacron, aramide, polyaramide, Kevlar®, Dyneema® or vegetable fibers (e.g. silk, sisal, hemp, etc.), for example.

In the sense of the present invention, a rod-shaped body is preferably made from a fiber-reinforced epoxy resin.

A rod-shaped body of this type is constructed preferably according to the rod-shaped bodies as described in WO 2009/141165 or WO 2007/000148 A2. In this respect, reference is made also to WO 2007/000148 A2.

By means of the tubular cannula of the cannula device, a rod-shaped body is exactly guided and aligned in the space until it is sheathed with the polymer.

The process of completely sheathing the rod-shaped body with polymer is carried out after it has left the discharge end of the cannula. In this way, the polymer is prevented from affecting the alignment of the rod-shaped body or moving the latter. This allows to exactly define its positioning in a medical instrument in the extruding process, whereby predetermined mechanical characteristics can be achieved. The rod-shaped body can be arranged with the aid of the cannula exactly at a definite position in the cross-sectional area of the medical instrument.

The molten polymer flows in the extrusion space approximately in laminar form along the cannula device which extends into the extrusion space. At the end of the cannula device, the polymer wets one or more rod-shaped bodies supplied by the cannula device, without exerting a moment on the rod-shaped body or bodies transverse to the manufacturing direction. It is preferred that the cannula device extends, as seen in the manufacturing direction, from the frontward end of the spindle sleeve by a distance of 3 mm or 5 mm, in particular at least 10 mm and particularly preferred by at least 15 mm into the extrusion space.

The cannula device allows in an easy way to adjust the distance between the discharge end of the cannula device and the discharge nozzle. Said distance is important, because the longer the time for which the rod-shaped body/bodies are dragged through the polymer melt in the extrusion space, the larger is the risk of a displacement of the rod-shaped body/bodies away from the desired position. On the other hand, the longer the distance is between the discharge end of the cannula device and the discharge nozzle, the more intense is the wetting of the rod-shaped body/bodies with the polymer melt.

Aligning and guiding the rod-shaped body is performed in the manufacturing direction beginning at the device for supplying rod-shaped bodies through the spindle sleeve into the extrusion space as far as to the area of the discharge nozzle. This allows a precise positioning of rod-shaped bodies in the space or in an exactly predefined position in a medical instrument.

Preferably, the cannula device can be adjusted in the spindle sleeve in the manufacturing direction without incurring a rotation of the cannula device about its longitudinal axis. This offers the possibility to freely change the distance between the discharge end of the cannula device and the discharge nozzle and to adapt it to the respective production conditions (transport speed of the rod-shaped bodies, flow velocity of the polymer melt, temperature, etc.) when the rod-shaped bodies are already threaded in the cannula device.

Due to the precise arrangement of at least one rod-shaped body in the medical instrument, the apparatus of the invention is able to produce medical instruments with predetermined mechanical characteristics such as elasticity, stiffness and breaking strength.

If a device (for instance disc-shaped) for guiding the rod-shaped body was provided instead of the cannula device, it would be required to fix it to the side wall of the housing of the extrusion device. A guiding device of such type would affect the uniform flow of the polymer and deflect it toward the rod-shaped body. In this case, it would be more difficult to cover the rod-shaped body with polymer in uniform manner. In addition, an irregular flow of the polymer would deflect the rod-shaped body if the latter was not arranged in a cannula in the extrusion space. Thus, it would not be possible any more to precisely arrange it at a predefined position in the medical instrument.

The spindle sleeve is designed for distributing and guiding the molten mass. On the other hand, it stabilizes the cannula of the cannula device arranged in it, so that its discharge end is maintained in straight alignment with the discharge nozzle despite the pressure of the polymer.

For distributing and guiding the molten mass and in order to bring the rod-shaped body in contact with the polymer, the spindle sleeve is formed so as to taper in the manufacturing direction; in particular, it has a conical design.

The cannula device may also comprise two or more tubular cannulas for supplying at least two rod-shaped bodies. It is also possible to provide three, four, five, six, seven, eight or even more cannulas. The cannulas may be concentrically arranged, with or without one or more central cannulas. An apparatus for extruding a medical instrument including a cannula device comprising at least two cannulas may also be designed such that the cannula device does not end in the extrusion space but in the spindle sleeve. This cannula device achieves a desired relative arrangement of the at least two rod-shaped bodies, with the possibility that the rod-shaped bodies can be arranged so as to be very close to one another. A preferred apparatus, however, is one in which the cannula device extends into the extrusion space, as the frontward end of the spindle sleeve, as seen in the manufacturing direction, has a substantially higher thickness than the cannula device. If the cannula device does not project into the extrusion space, a discontinuity in the flow speed occurs due to the abruptly changing cross-sectional area of the flow of the polymer melt in the area of the end of the spindle sleeve. Undesired turbulences in the flow of the polymer melt are produced, too.

Owing to the provision of two or more tubular cannulas, the apparatus according to the invention is capable of providing medical instruments with two or more rod-shaped bodies.

By means of the one or more tubular cannula(s) of the cannula device, the rod-shaped bodies are exactly guided and aligned relative to one another until they are sheathed with the polymer.

This means that the rod-shaped bodies are figured or arranged in the medical instrument corresponding to the pattern or arrangement of the cannulas of the cannula device. The exact position of the rod-shaped bodies in the medical instrument is determined by the cannula device, in particular through the relative positioning of the individual cannulas with respect to one another. Hence, the arrangement or the position of the cannulas defines the position of the rod-shaped bodies in the medical instrument.

The process of completely sheathing the rod-shaped bodies with polymer preferably occurs not until they have left the discharge end of the cannula. Due to the guidance of the rod-shaped bodies in the cannulas up into the area behind the spindle sleeve as seen in the manufacturing direction, the polymer is prevented from deflecting the rod-shaped bodies too much and changing their mutual arrangement. This allows to arrange the rod-shaped bodies in the cross-section of a medical instrument in a predefined position.

The stiffness of the medical instrument can be influenced by the number of the rod-shaped bodies and their distance from the center of the medical instrument. In other words, the higher the number of the rod-shaped bodies which are arranged in the medical instrument and the larger their distance from the center, the higher is the stiffness of the medical instrument. Further, the stiffness can be controlled via the diameter of the rod-shaped bodies and/or the polymer which is used.

The rod-shaped body is aligned and guided in the manufacturing direction up to shortly before the discharge nozzle. This is why the guidance via the cannula device does not impair the flow of the molten mass; vice versa, the rod-shaped bodies are not deflected by the melt flow out of their predefined position.

The elongated cannulas further achieve a guidance of the rod-shaped bodies where only small forces act on the rod-shaped bodies, which is due to the large inner surface or guiding surface of the cannulas. If short ring portions were used for guiding, these would have a negative impact on the flow of the molten mass.

The guidance by means of the cannula device also avoids in a safe and reliable manner that individual rod-shaped bodies protrude from the medical instrument after having been covered with the polymer. This risk exists due to the small outer diameter of the medical instrument, if there was no cannula device or guidance for the rod-shaped bodies, as the latter would change their position during the extrusion process because of the pressure of the molten mass. The process of sheathing the one or more rod-shaped bodies begins when they leave the cannula device.

Preferably, provision is made that the number of the cannulas of the cannula device is equal to the number of the rod-shaped bodies to be provided in the medical instrument; alternatively, the number of the cannulas is by one larger than the number of the rod-shaped bodies. The number of the cannulas will be discussed in more detail below. Due to using several cannulas, it is possible to arrange the rod-shaped bodies in the medical instrument in the desired geometry.

Further, the supply end of a cannula may be designed so as to be funnel-shaped or expanded contrary to the manufacturing direction, in order to avoid any rubbing or abrading action of the rod-shaped body at the supply end of the cannula and to simplify the supply and in particular the insertion of the rod-shaped bodies. In the area between the device for supplying rod-shaped bodies and the supply end of the cannula device, a supply device may be arranged which is preferably designed as a supply disc and comprises at least one guiding hole for guiding a rod-shaped body.

Due to the fact that a rod-shaped body is arranged in the guiding hole, the supply disc absorbs the vibrations of the one or more rod-shaped bodies, which vibrations are transmitted to the rod-shaped bodies during the supply process due to the structure of the device for supplying rod-shaped bodies, so that they can be supplied to a cannula of the cannula device in an almost vibration-free state.

Further, the guidance in the guiding hole of the supply disc allows to supply a rod-shaped body to the extrusion device with uniform tension, simplifying the positioning of the rod-shaped body in the medical instrument or at a predetermined point.

In addition, the supply disc ensures a pre-adjustment of the geometry of the rod-shaped bodies for their positioning in the medical instrument before they are supplied to the cannula device and then to the extrusion space.

The supply disc prevents a rod-shaped body from exerting a rubbing or abrasive effect on the cannula inlet and being damaged and/or made to oscillate or being biased with an additional tension. Such rubbing effect is caused by the movements on the device for supplying rod-shaped bodies and of the material loom and its coils. The supply disc thus inhibits vibrations of the delicate rod-shaped bodies.

The supply disc comprises one guiding hole for each rod-shaped body to be supplied, i.e. the supply disc may comprise two or more guiding holes.

In the area between the discharge end of a cannula and the discharge nozzle, a centering device or centering disc may be arranged, which comprises at least one centering hole to supply a rod-shaped body to the discharge nozzle in a predetermined position.

The centering hole of the centering disc accomplishes a pre-adjustment of the diameter of the medical instrument and/or of the position of the rod-shaped body/bodies in the medical instrument, i.e. the centering hole defines the position of one or more rod-shaped bodies.

In case several rod-shaped bodies are provided, they can be converged through the centering hole of the centering disc so as to be closer to one another, whereby the distances between the rod-shaped bodies are made smaller. Thus, it is an important point that the diameter of the centering hole is adapted to the desired mechanical characteristics and/or the external dimensions of the medical instrument.

Preferably, the centering hole can be formed so as to taper in the manufacturing direction, and at the end in the manufacturing direction it may have a diameter which amounts to approximately 50% to 80% or 100% of the diameter of a melt channel of the discharge nozzle.

The process of inserting the rod-shaped body into the centering hole is facilitated by the centering hole tapering in the manufacturing direction.

In case of providing several rod-shaped bodies, provision can also be made that the interspaces between the rod-shaped bodies within the medical instrument will be filled with polymer while passing through the centering hole, whereby they are embedded and kept together in the polymer. The process of embedding the rod-shaped body in the polymer normally occurs already in the area upstream of the centering device.

The above-mentioned compression of the rod-shaped bodies may be required in particular if the distance of the individual rod-shaped bodies relative to one another is supposed to be smaller than the added wall thicknesses of the cannulas of the cannula device.

In the course of passing the rod-shaped bodies through the discharge nozzle, the final external sheathing of the rod-shaped bodies with the polymer is made and the final outer geometry of the medical instrument is determined through the shape and dimension of the cross-section of the melt channel of the discharge nozzle. In this way, a medical instrument is made available in which the rod-shaped bodies are precisely arranged in the space or in the medical instrument at a defined distance. A crucial point for this production step is the distance from the discharge end of the cannula device and/or the centering disc to the discharge nozzle, as seen in the manufacturing direction. The distance between the centering disc and the discharge nozzle in the manufacturing direction is between 1 mm and 60 mm or between 25 mm and 35 mm.

In addition to the devices described above, the outer diameter of the medical instrument is determined by the diameter of the melt channel of the discharge nozzle, the pressure of the polymer in the extrusion space and the extrusion speed. Further, the length of the melt channel, the temperature of the molten mass and the withdrawal speed may also have an influence on the dimensions of the medical instrument, as the polymer possesses specific shape memory characteristics.

The discharge nozzle may also be preferably designed in such a manner that the positioning of the melt channel can be adjusted transverse to the manufacturing direction.

In the area between the supply disc and the supply end of the cannula device, an alignment device may be arranged, which preferably is designed as an alignment disc comprising at least one alignment hole for aligning a cannula of the cannula device. The alignment device is arranged in the area of a supply end of a cannula of the cannula device. It is formed in such a manner that it receives a supply end of a cannula in an alignment hole. In this way, a cannula of the cannula device is stabilized and also aligned in the direction of the supplied rod-shaped body, so that the angle relative to the supplied rod-shaped body becomes more acute.

Opposite the manufacturing direction, the supply ends of the cannula device are expanded preferably by an angle of 3° to 30° and in particular of 5° to 15°.

In manufacturing direction downstream of the discharge nozzle, a cooling device, in particular a water bath, may be arranged for cooling the medical instrument.

The polymer embedding and/or surrounding the rod-shaped body/bodies is hardened in the water bath.

Following the water bath in the manufacturing direction, a roller device may be provided which is designed for aligning the medical instrument. The adjustment of the positioning of the rod-shaped bodies in the medical instrument is also accomplished via the position of the melt channel of the discharge nozzle and via the roller device. In particular, the centering disc forms a fixed point and the roller device forms a further fixed point, whereby the medical instrument is kept taut between these two points and hence its positioning in the apparatus can be adjusted.

By means of one or more of the devices as described above for guiding and/or aligning the rod-shaped bodies, the latter can be deflected in such a manner that they are present in the desired arrangement on the one hand, and have their outer side uniformly sheathed with polymer, on the other hand.

Moreover, the rod-shaped bodies can be kept under tension from the centering device to the roller device, in order to align the melt channel of the discharge nozzle relative to the rod-shaped bodies in space, so that an optimum final product can be made available in which the rod-shaped bodies are completely sheathed with polymer and assume a predetermined position in space.

Further, in the manufacturing direction downstream of the cooling device, a withdrawal device may be arranged which is designed to discharge the medical instrument from the apparatus and/or to guide it through the apparatus.

The withdrawal device is able to ensure that the medical instrument and in particular at least one rod-shaped body arranged therein are permanently kept under tension, so that they do not sag, guaranteeing a constant diameter over the entire length of the medical instrument and a uniform positioning of the rod-shaped body in the medical instrument.

Preferably, the withdrawal device is realized as a crawler-type withdrawal device provided with chain-like or profiled elements, or as a band-type withdrawal device comprising transport bands. As an alternative, a drum-like withdrawal device, a linear withdrawal means comprising carriages or a roller-type withdrawal device may be provided. The withdrawal device is realized in particular such that the medical instrument is solidified and/or stretched by the withdrawal process as little as possible, so that the shape of the medical instrument is not changed through the withdrawal means, if possible.

The cannula device may comprise an adjustment device through which the position of the cannula device can be adjusted in the manufacturing direction. The adjustment device preferably is a thread surrounding the cannula device. The adjustment device allows to exactly adjust the position of at least one cannula in the manufacturing direction and hence the distance from the discharge ends of the cannulas to the centering device or discharge nozzle.

The distance between the discharge end of the cannula and the centering disc or the discharge nozzle, i.e. the area in which at least one rod-shaped body is arranged in the extrusion space without any guidance, is crucial for maintaining a constant geometry of the catheters and guide wires, both with respect to the positioning of the rod-shaped body in the medical instrument and the external dimensions and the geometry of the medical instrument. The distance from the discharge end of the cannula to the centering disc or discharge nozzle must not be too large, so as to prevent an excessively high deflection of the rod-shaped bodies through the polymer, and it must not be too small, either, so that the interspaces between the rod-shaped bodies are completely filled up with polymer. The distance between the discharge end of the cannula to the centering disc or the end of the discharge nozzle is in the range from 0 mm or 0.1 mm to 30 mm, or from 1 to 15 mm and preferably from 2 mm to 5 mm.

It is preferred that the device for supplying rod-shaped bodies is designed as a material loom which has braked coils or rollers arranged thereon, which have the rod-shaped bodies wound up on them. Due to decelerating the coils, fewer vibrations are produced in the rod-shaped bodies during the unwinding process and they are kept under constant tension.

At least the side wall of the extrusion device may be provided with a heating device.

The heating device allows to keep the extrusion space at a constant temperature in order to keep the polymer in a state in which it is capable of flowing. Further, a throttle or discharge valve may be provided on the extrusion space, to discharge polymer from the extrusion space. This will prevent any degraded polymer from being processed; on the other hand, discharging the polymer offers the possibility to keep it in motion or to accelerate it in order to avoid stagnation.

The cannula device may comprise a central cannula for supplying air and several decentralized or peripheral cannulas for supplying rod-shaped bodies; in this case, the central cannula is provided with a device for supplying compressed air to build up a supporting air pressure during the extrusion for forming a catheter lumen in the medical instrument which in particular is a catheter.

A cannula device of this type makes it possible to extrude catheters containing rod-shaped bodies which are embedded in the catheter wall.

The arrangement of the peripheral cannulas defines the position of the rod-shaped bodies regarding the lumen and their relative arrangement with respect to one another and hence their position in the catheter wall.

The discharge ends of one or more cannulas may protrude forward from the spindle sleeve in the manufacturing direction. Preferably, one or more cannulas protrude from the spindle sleeve by 1 mm to 20 mm or 7 mm to 10 mm.

The apparatus according to the invention for extruding of a medical instrument which can be inserted into a human or animal body is distinguished in that a medical instrument within the apparatus and in particular in the cannula device has multiple contact with it. This results in a multitude of contact points between the apparatus according to the invention and the medical instrument. In this way, the force exerted on the medical instrument during the production process is evenly distributed over the outer surface of the medical instrument, whereby the medical instrument will not experience any deformation.

The apparatus according to the invention may be designed for the parallel production of several medical instruments.

According to the invention, a method is also provided for extruding a medical instrument which can be inserted into a human or animal body, in which a rod-shaped body is supplied in a manufacturing direction by means of a device for supplying rod-shaped bodies to an extrusion device and is covered with a polymer in the extrusion device, the medical instrument receiving its final shape while leaving a discharge nozzle of the extrusion device in the manufacturing direction. The rod-shaped body is guided by means of a tubular cannula of a cannula device as far as to an extrusion space of the extrusion device up into the area in front of the discharge nozzle as seen in the manufacturing direction, so that it can be arranged in a predefined position in the medical instrument.

The invention will be explained in more detail below on the basis of the drawings in which:

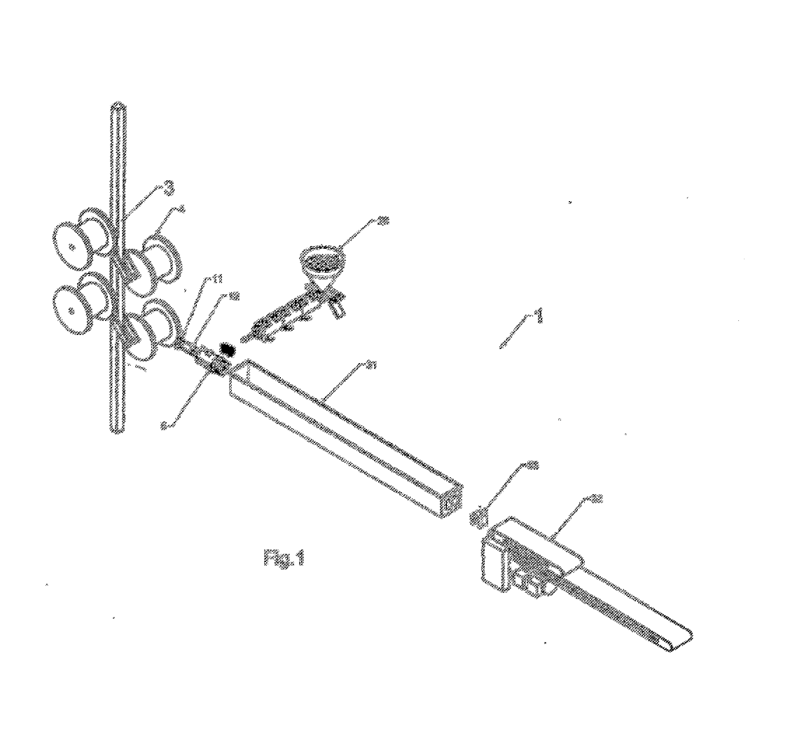

FIG. 1 is an illustration of an apparatus according to the invention for extruding medical instruments, in a perspective view,

FIG. 2 is a schematic illustration of an apparatus according to the invention for extruding medical instruments, in a longitudinal section,

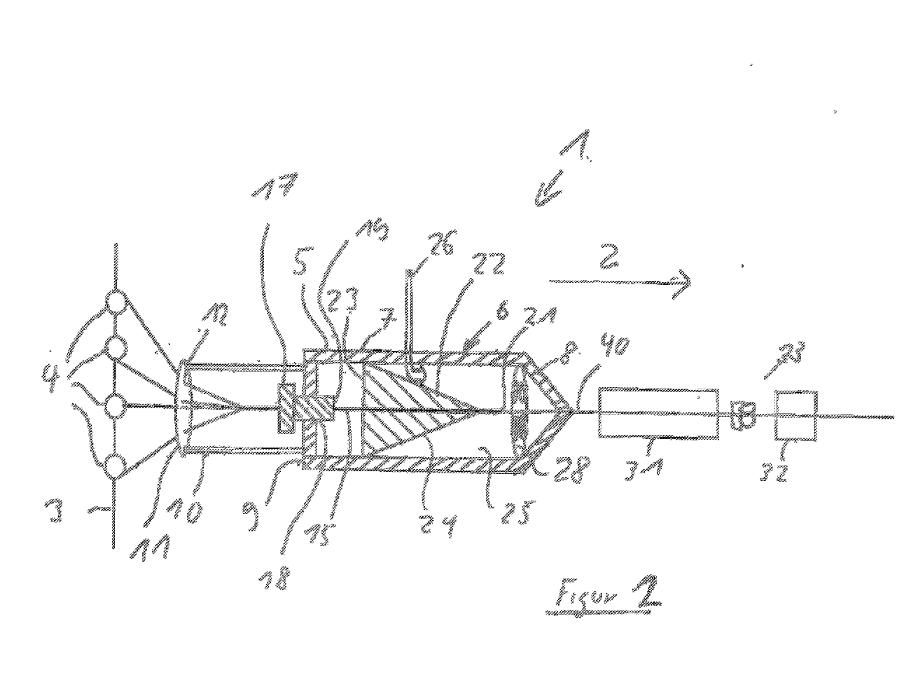

FIG. 3 shows an extrusion device of the apparatus from FIG. 1, in particular for manufacturing guide wires, in a laterally sectioned view,

FIG. 4 shows the extrusion device from FIG. 2 in a side view,

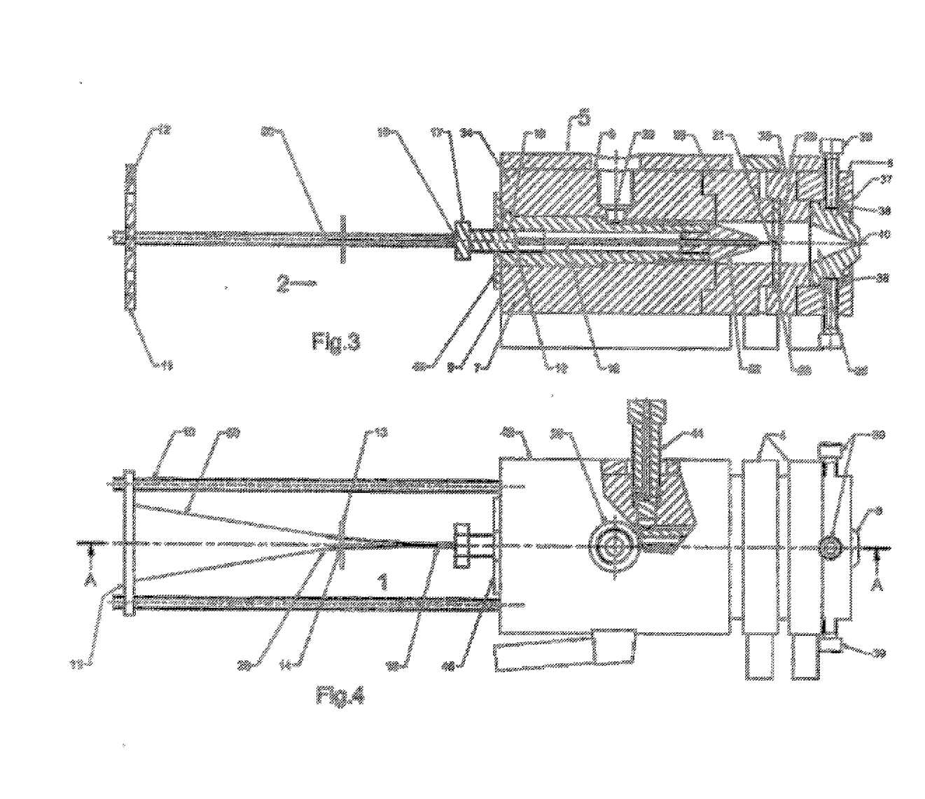

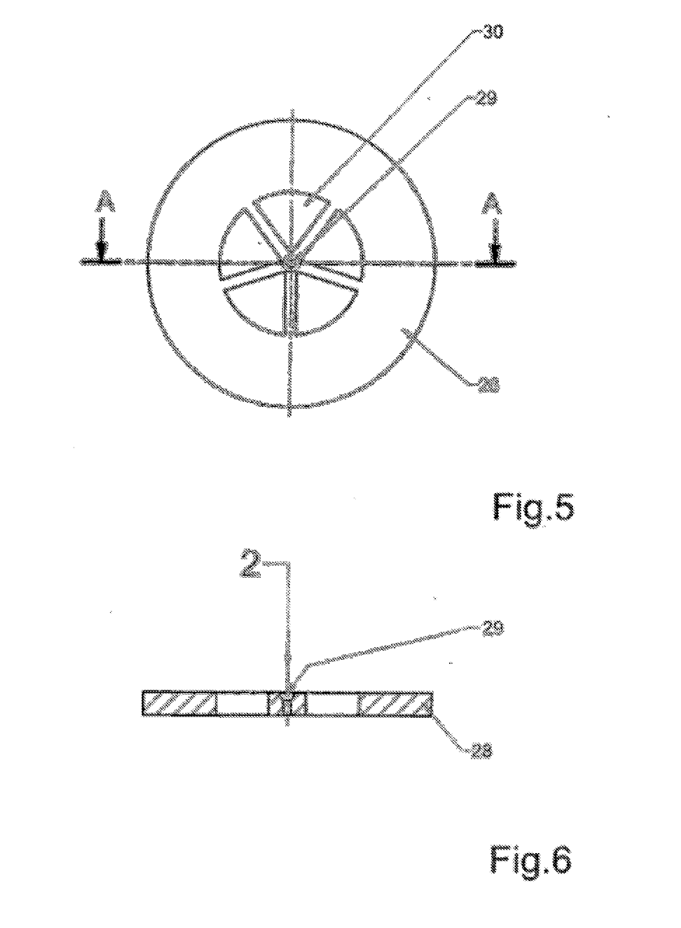

FIG. 5 shows a centering disc according to the invention in a top view as seen from the front,

FIG. 6 shows the centering disc from FIG. 5 in a cross-section taken along line A-A, comprising a centering hole,

FIG. 7 is an exploded view of the extrusion device shown in FIG. 3,

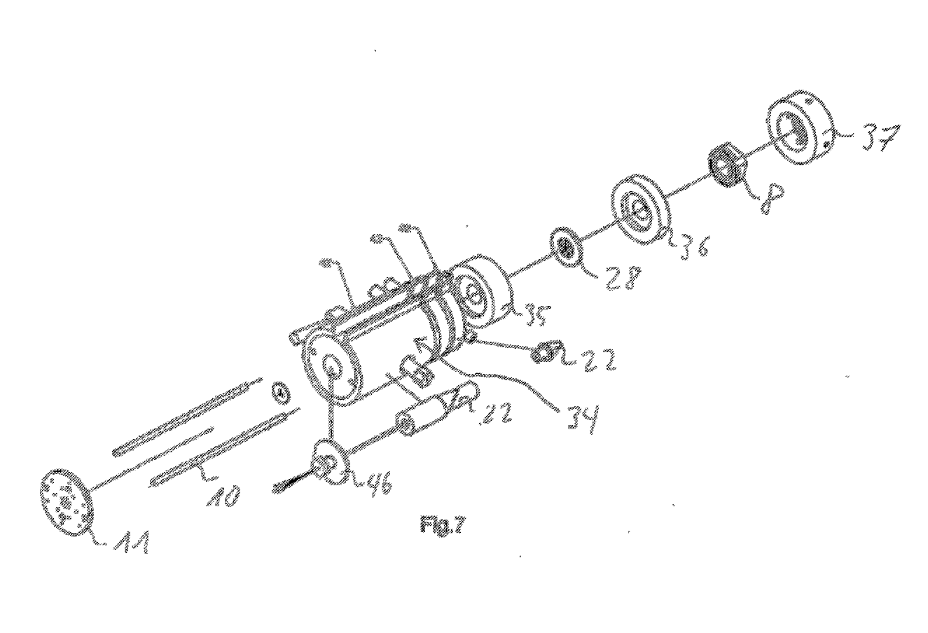

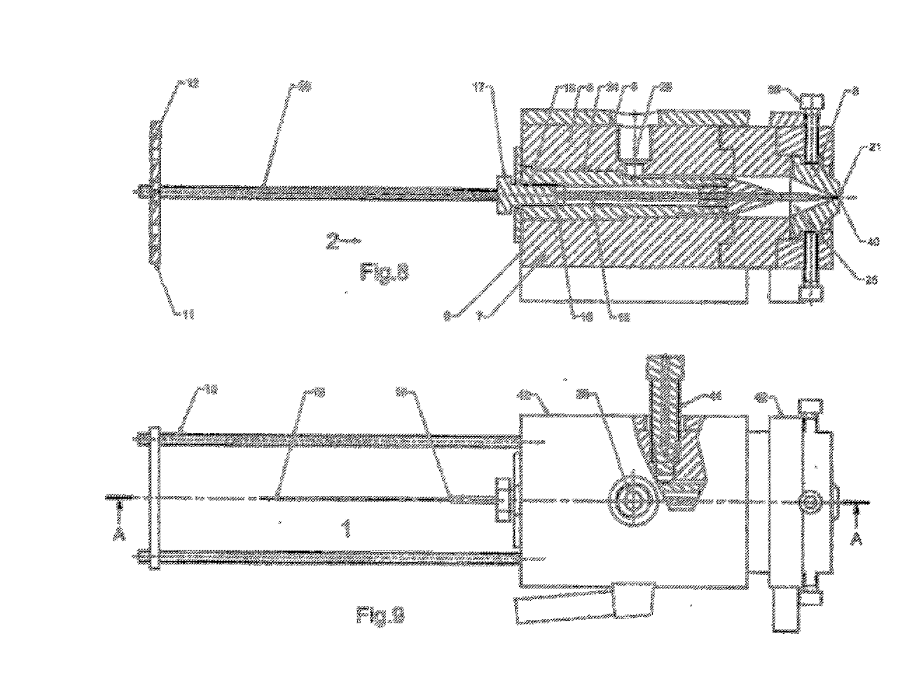

FIG. 8 is a further exemplary embodiment of an apparatus according to the invention, in particular for manufacturing catheters, in a laterally sectioned view,

FIG. 9 shows the extrusion device from FIG. 8 in a side view,

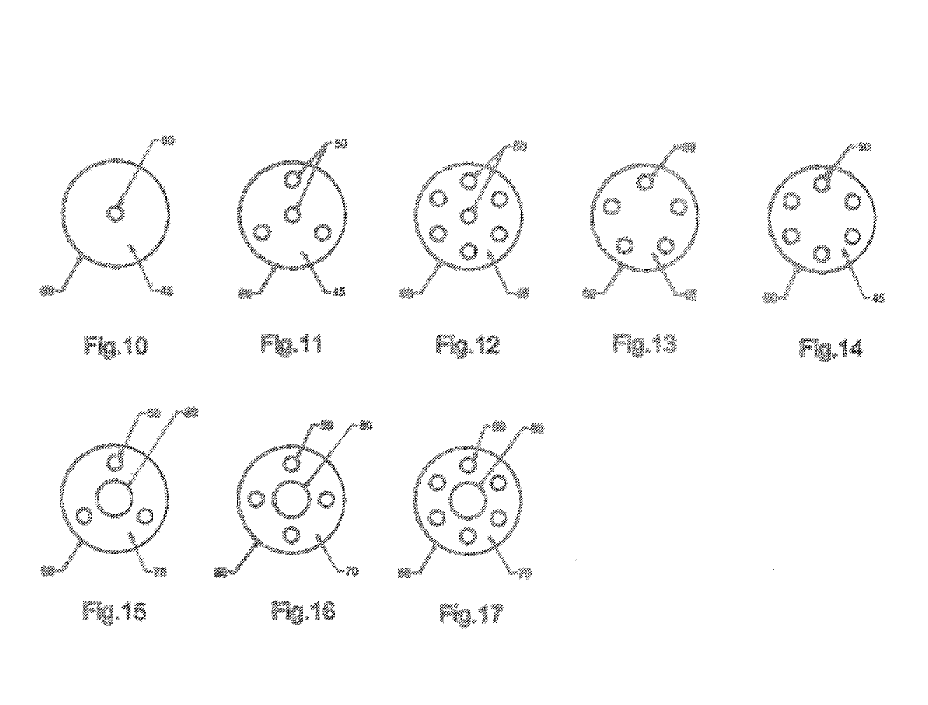

FIG. 10 is a cross-section of a guide wire which can be manufactured with the apparatus according to the invention and comprises a central rod-shaped body,

FIG. 11 is a cross-section of a guide wire which can be manufactured with the apparatus according to the invention and comprises a central rod-shaped body as well as three rod-shaped bodies which are each arranged with the same radial distance,

FIG. 12 is a cross-section of a guide wire which can be manufactured with the apparatus according to the invention and comprises a centrally arranged rod-shaped body as well as six rod-shaped bodies surrounding the central rod-shaped body and being arranged so as to be equally spaced from each other,

FIG. 13 is a cross-section of a guide wire which can be manufactured with the apparatus according to the invention and comprises five rod-shaped bodies which are arranged so as to be equally spaced from each other,

FIG. 14 is a cross-section of a guide wire which can be manufactured with the apparatus according to the invention and comprises six rod-shaped bodies which are each arranged with the same radial distance,

FIG. 15 a cross-section of a catheter which can be manufactured with the apparatus according to the invention and comprises a catheter lumen as well as three rod-shaped bodies embedded in the catheter wall and surrounding said catheter lumen with identical radial and mutual distances,

FIG. 16 a cross-section of a catheter which can be manufactured with the apparatus according to the invention and comprises a catheter lumen as well as four rod-shaped bodies embedded in the catheter wall and surrounding said catheter lumen with identical radial and mutual distances,

FIG. 17 a cross-section of a catheter which can be manufactured with the apparatus according to the invention and comprises a catheter lumen as well as six rod-shaped bodies embedded in the catheter wall and surrounding said catheter lumen with identical radial and mutual distances,

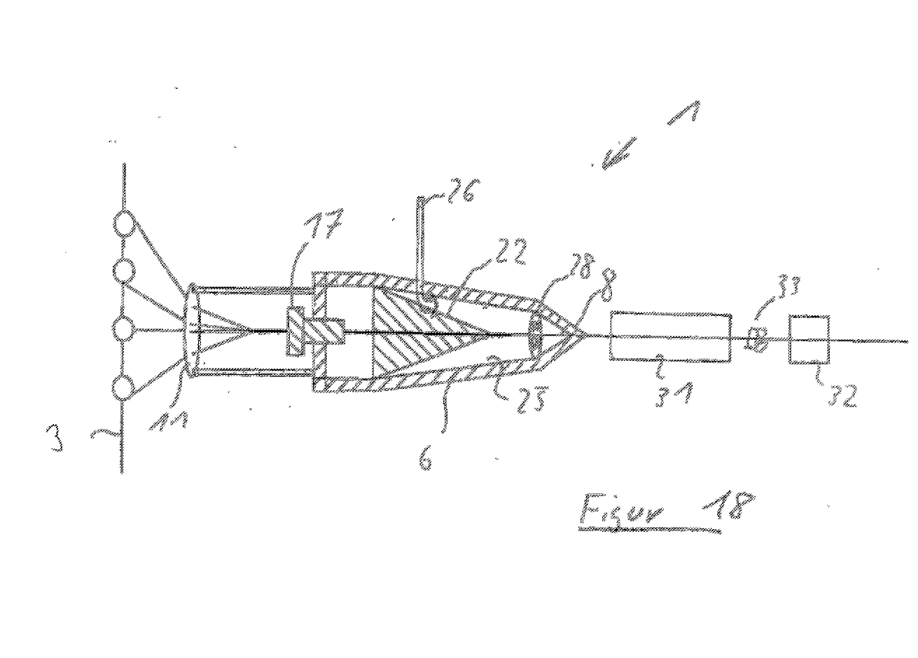

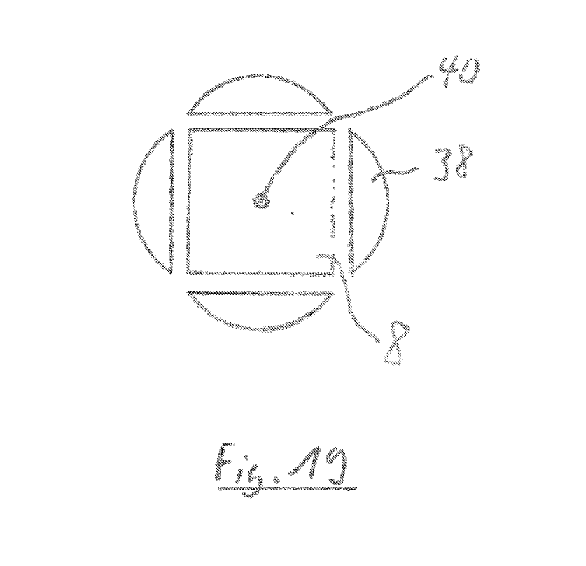

FIG. 18 is a schematic illustration of a further apparatus according to the invention for extruding guide wires and catheters, in a longitudinal section, and

FIG. 19 shows a discharge nozzle in a top view.

According to the invention, an apparatus 1 is provided for extruding a medical instrument which can be inserted into a human or animal body (FIG. 1 to FIG. 7).

As seen along a manufacturing direction 2, the apparatus 1 comprises a device 3 for supplying rod-shaped bodies 50, an extrusion device 5, a cooling device 31, a roller device 33 and a withdrawal device 32 (FIG. 1).

By way of example, the apparatus is described on the basis of an apparatus 1 for producing a medical instrument, which is a guide wire 45 with four rod-shaped bodies 50 arranged therein, wherein one rod-shaped body is centrally arranged and the other three peripheral rod-shaped bodies are arranged with equal radial and mutual distances and surround the central rod-shaped body.

It is preferred that the device 3 for supplying rod-shaped bodies 50 is a material loom. Four coils 4 are arranged on the material loom 3 one above the other, side by side and/or so as to be offset from one another. The rod-shaped bodies 50 are wound up on the coils 4. The coils 4 have one brake each in order to control the withdrawal speed and damp any vibrations. It is by means of the brakes (not illustrated) that the rod-shaped bodies 50 are kept taut.

The device 3 for supplying rod-shaped bodies 50 is designed for supplying rod-shaped bodies 50 to an extrusion device 5.

The extrusion device 5 will be explained in more detail in the following.

The schematic structure of the extrusion device 5 comprises a housing 6 with a surrounding side wall 7 which, at the frontward end as seen in the manufacturing direction 2, is provided with a nozzle wall comprising a discharge nozzle 8 and, at the rearward end as seen in the manufacturing direction, is provided with a supply wall 9 (FIG. 2).

The supply wall 9 is provided with at least two threaded rods 10 extending contrary to the manufacturing direction 2 and intended for receiving a supply device 11. The position of the supply device along the manufacturing direction 2 can be adjusted via the threaded rods.

The supply device 11 is arranged so as to be situated substantially transverse to the manufacturing direction 2. At the outer periphery, the supply device 11 comprises several holes in a radially surrounding fashion, which serve for fastening the supply device with nuts on the threaded rods. The supply device 11 preferably is a disc-shaped plate which in the present exemplary embodiment is provided with four guiding holes 12, with each guiding hole 12 being designed for guiding one rod-shaped body 50. Preferably, the edges of the guiding holes 12 are designed so as to be rounded off. The position of the guiding holes 12 relative to the manufacturing direction 2 can be adjusted via the holes at the outer periphery of the supply device 11.

The guiding holes 12 accomplish a first alignment of the individual rod-shaped bodies 50 in the manufacturing direction 2. Any vibrations generated during the supply by the material loom 3 or due to the unwinding process are attenuated by being supported received in the guiding holes 12; thus, the rod-shaped body 50 can be supplied to an alignment device 13 in a state in which they are virtually free from vibrations.

The coils 4 of the material loom are arranged on the material loom 3 in a compact manner such that the rod-shaped bodies can be supplied to the supply device 11 with an angle which is as flat as possible.

As seen in the manufacturing direction 2, an alignment device 13 is provided behind the supply device 11. The alignment device 13 is also a disc-shaped element which is arranged substantially transverse to the manufacturing direction 2.

The alignment disc 13 comprises four alignment holes 14.

One alignment hole is arranged to be central in the alignment disc and the other three alignment holes are peripherally arranged with equal radial distances from the central alignment hole. The alignment holes 14 are each designed for receiving one cannula 15 of a cannula device 16 and are connected thereto.

The cannula device 16 comprises an adjustment device 17. The adjustment device 17 may be designed as a screw, for example, whose external thread can be received in a corresponding threaded hole 18 in the supply wall 9. The adjustment device 17 comprises a through-hole 19 extending in the manufacturing direction 2. The position of the cannulas 15 in the manufacturing direction can be adjusted via the adjustment device.

According to the present exemplary embodiment, four tubular cannulas 15 in a cannula tube 16 are arranged in the through-hole 19. The cannulas 15 each have a supply end 20 contrary to the manufacturing direction 2 and a discharge end 21 pointing in the manufacturing direction. The cannula tube 16 is fixed in the through-hole 19 of the adjustment device by means of welding or soldering, for instance.

Preferably, the soldered joint at the transition from the cannula tube 16 to the cannulas 15 is designed in such a manner that there is an angle of 45° so as to not impair the melt flow.

The supply ends 20 associated to the peripheral cannulas and oriented contrary to the manufacturing direction 2 are arranged in the alignment holes 14 of the alignment device 13 such that they radially diverge contrary to the manufacturing direction 2 with a flat angle. Owing to such an outward alignment, the angle with respect to the rod-shaped body to be supplied is designed to be more acute.

Arranged in the housing 6 is a conical spindle sleeve 22 which tapers in the manufacturing direction 2. The spindle sleeve 22 comprises a base wall 23 and a jacket wall 24. The jacket wall 24 adjoins the side wall 7 of the housing 6 in tight fashion. The spindle sleeve 22 may comprise a cylinder-shaped portion extending from the base wall 23 contrary to the manufacturing direction 2 and adjoining the jacket wall 24 of the housing 6 in tight fashion. Said portion is connected to the housing in a torque-proof manner via a radial dowel pin and a corresponding milling groove.

The cannulas 15 are supported in the adjustment device 17 in a preferably rotatable manner and are guided in the spindle sleeve such that they are not able to rotate therein. This allows to change the position of the cannulas 15 in the manufacturing direction by rotating the adjustment device without incurring a rotation of the cannulas in this process.

The space in the housing 6 between the base wall 23 the spindle sleeve 22, the side wall 7 and the discharge nozzle 8 defines an extrusion space 25.

In the area of the extrusion space 25, a polymer supply device 26 is attached to the housing. The polymer supply device 26 comprises a melt pump or a conveyor screw device to pump liquefied polymer into the extrusion space 25. The polymer is a polymer which is similar to the polymers described in the introductory portion of the description for covering rod-shaped bodies.

The housing 6 of the extrusion device, in particular the side wall 7 and/or the discharge nozzle 8, are provided with a heating device 42 (FIG. 4).

The cannulas 15 of the cannula device 16 as well as the cannula tube pass through a through-hole 27 provided in the spindle sleeve 22 and extending in the manufacturing direction. The cannula tube 16 is designed such that the cannula device 16 can be pulled out of the spindle sleeve contrary to manufacturing direction. The discharge ends 21 of the cannulas 15 of the cannula device 16 end in the extrusion space 25.

At a distance of approximately 1 mm to 20 mm and preferably 7 mm to 10 mm remote from the discharge ends 21 of the cannulas 15 as seen in the manufacturing direction 2, a centering device 28 is provided in the extrusion space 25 substantially transverse to the manufacturing direction 2 (FIG. 2, FIG. 3).

The centering device 28 is a disc-shaped element approximately having the shape of a cartwheel. The centering disc 28 comprises in its center a centering hole 29. The centering hole 29 tapers in the manufacturing direction 2. Further, e.g. three to five approximately triangular melt openings 30 are arranged around the centering hole 29.

When passing through the centering disc 28, the rod-shaped bodies 50 are wetted with polymer. Due to moving and deflecting the melt flow in the area of the centering hole 29, the rod-shaped body is wetted with polymer in optimum manner. Any polymer adhering to the surface of the rod-shaped bodies is entrained in the manufacturing direction.

When passing through the centering disc 28, the rod-shaped bodies 50 may also be aligned in such a manner that they are converged to a higher degree within the polymer sheathing so as to be present in the required geometry. In addition, owing to the centering hole, the geometry of the guide wire 45 is stabilized and especially the space between the rod-shaped bodies is filled with the molten mass.

The outer dimensions of the guide wire 45 are defined, inter alia, by the diameter of the melt channel of the discharge nozzle.

The extrusion space may also be provided with a throttle 44 (FIG. 4) to discharge polymer from the extrusion space. This measure prevents the polymer from remaining too long in the extrusion space and degrading there. Moreover, an opened throttle is able to increase the flow speed of the polymer. Due to the fact that the throttle 44 is arranged at the end of the extrusion space which is contrary to the production direction, the linear flow of the melt flow is not affected by the discharge process.

A cooling device 31 is provided in the area downstream of the discharge nozzle 8 as seen in the manufacturing direction 2. The cooling device 31 is preferably designed as a water bath through which the guide wire 45 is conveyed whereby it is cooled and hardened.

Behind the cooling device 31 as seen in the manufacturing direction, a roller device 33 for guiding the medical instrument is arranged. The roller device comprises e.g. two pairs of rollers which are arranged so as to be offset by 90° relative to each other and designed for deflecting and guiding a medical instrument (FIG. 1).

Downstream of the water bath 31 as seen in the manufacturing direction 2, a withdrawal device 32 is arranged. The withdrawal device 32 is preferably designed as a band-type withdrawal means.

Such band-type withdrawal means are known from profile extrusion technology.

FIGS. 3 and 4 show the structure of the housing in more detail. There is to be seen a housing 6 of modular construction, whose side wall 7 is formed from a supply portion 34, two fixing portions 35, 36 and an extrusion portion 37.

The discharge nozzle 8 comprises four circular segments 38 whose position can be adjusted transverse to the manufacturing direction 2 via a set screw 39 each (FIG. 19). The set screws 39 and the circular segments 38 allow to adjust the position of the discharge nozzle 8 and hence of the melt channel 40 transverse to the manufacturing direction 2. Due to the fact that the circular segments 38 are formed so as to be separate from the discharge nozzle 8, the circular segments 38 may be provided with threaded holes for receiving the set screws 39. Upon actuating the set screws, the discharge nozzle slides along the straight faces of the circular segments 38 to the desired position.

According to FIGS. 3 and 4, the cannula device 16 is screwed in a tubular spindle sleeve portion 41, whereby the position of the cannula device 16 can be adjusted in the manufacturing direction 2. The spindle sleeve portion 41 is fixed in the supply portion 34. Arranged on the front end of the spindle sleeve portion 41 is the spindle sleeve tip 22. The spindle sleeve portion 41 is fixed in the supply portion 34 of the housing 6 by means of a fastening disc.

As is shown in detail in FIG. 3, the centering device 28 is arranged and clamped between the two fixing portions 35, 36 of the housing 6.

A method of the invention for manufacturing guide wires or catheters for insertion into a human or animal body will be exemplarily explained below on the basis of a guide wire which has four rod-shaped bodies arranged therein.

According to the method of the invention, for instance four rod-shaped bodies 50 are supplied from respective coils 4 which are arranged on a device 3 for supplying rod-shaped bodies 50 through four centering holes of a supply device 11 toward the supply ends of four cannulas of a cannula device 16.

The four rod-shaped bodies are supplied to the extrusion space 25 through the cannulas 15 of the cannula device 16 in the manufacturing direction right through the spindle sleeve 22.

A polymer mass in a state in which it is capable of flowing is supplied to the extrusion space 25 via a laterally arranged polymer supply device 26.

The housing 6 of the extrusion device 5 as well as the discharge nozzle are heated by means of a heating device in order to keep the polymer mass in a state in which is capable of flowing.

The individual rod-shaped bodies 50 come into contact with the molten mass while leaving the discharge ends 21 of the cannulas 15. In this process, the interspaces of the individual rod-shaped bodies 50 are filled with polymer from the extrusion space, whereby they are embedded in the polymer and glued to each other.

During the subsequent process of passing the rod-shaped bodies 50 through a central centering hole 29 of the centering device 28, the relative movement between the molten mass and the rod-shaped bodies results in wetting the rod-shaped bodies with the molten mass in optimum fashion. Moreover, the centering hole stabilizes the mutual arrangement of the rod-shaped bodies and maintains it.

If it happens that the distance of the rod-shaped bodies in the cannulas of the cannula device is larger than the distance which they are supposed to have in the medical instrument, provision is made that the diameter of the centering hole is designed to be such that the rod-shaped bodies are compressed while passing through the centering hole. The compressing of the rod-shaped bodies may be required in particular if the distance of the individual rod-shaped bodies relative to one another is to be smaller than the added wall distances of the cannulas of the cannula device.

A point which is important for this production step is the distance from the end of the cannulas up to the discharge nozzle, as said distance determines the time for which the rod-shaped bodies are contacted with the molten mass.

The final determination of the geometry of the medical instrument occurs while leaving the discharge nozzle 8, which is defined by the diameter of the melt channel of the discharge nozzle.

Subsequently, the guide wire which has been produced in this way is cooled in the water bath.

Beginning at the centering device, a roller device 33 deflects the rod-shaped bodies in such a manner that they are affected by the polymer melt as little as possible and can be uniformly sheathed with polymer.

The process of conveying the rod-shaped bodies 50 through the extrusion device 5 is achieved by means of a band-type withdrawal device. The withdrawal speed of the band-type withdrawal device dictates the speed with which the medical instrument is produced by the extrusion device 5.

An apparatus of the invention according to a second exemplary embodiment (FIG. 8, FIG. 9) is designed substantially as the first exemplary embodiment. This apparatus 1 is realized without a centering device 28. With this apparatus, the second fixing portion 36 may be dispensed with, as there is no need to provide a centering device in the housing 6. An apparatus of this type is especially suited for manufacturing catheters.

With this apparatus 1, the cannulas 15 extend as far as into the area of the discharge nozzle 8 a short distance ahead of the melt channel 40, with the central cannula for providing the supporting air pressure terminating in the manufacturing direction preferably flush with the end of the melt channel.

Preferably, the apparatus according to the invention is designed for manufacturing guide wires with one central and two to ten and preferably three to six peripheral rod-shaped bodies, for example. In accordance with the mechanical demands imposed on the medical instrument, one central rod-shaped body and several peripheral rod-shaped bodies may be provided. Basically, any arrangement of the rod-shaped bodies with or without a central rod-shaped body is possible (FIG. 10 to FIG. 14).

The apparatus according to the invention may also be designed for manufacturing catheters with 3 to 10 rod-shaped bodies, for instance, which are arranged in the catheter wall (FIG. 15 to FIG. 17).

Further technical details of the apparatus according to the invention will be explained in the following.

It is also possible that the spindle sleeve is realized—at least in portions—with a cross-section which is unchanged or constant in the manufacturing direction.

The spindle sleeve is supposed to distribute the melt flow (which is supplied under an angle of preferably 90°) uniformly across a ring-shaped outlet cross-section. However, other angles are also conceivable.

In most cases, the molten mass is supplied from the extruder or the polymer supply device to the extrusion device under an angle of 90°. A distributor channel may be provided which converts the radially moving melting into an axially flowing, tubular melt flow. To this end, for instance a coat hanger distributor may be provided, as it is known from the field of wide-slot tools. With such distributor, the distributor channel lies in a plane and looks like a coat hanger when seen from the top. The use of a cardioid distributor is also conceivable (FIG. 7). Basically, most different types of spiral mandrel distributors which are known from prior art can be transferred to the spindle sleeve of the apparatus according to the invention, in which the flow of the polymer is made to rotate by means of screw-shaped grooves or grooves of similar design. Generally, it has to be made sure that the flow profile around the spindle sleeve is determined in such a manner that the flow downstream of the deflection area has the same average discharge velocity across the entire spindle sleeve. To this end, it is required that the flow resistances on all melt paths from the feed area to the outlet opening are equal.

According to the invention, the molten mass should be accelerated in the extrusion space in order to suppress a stagnation of the polymer.

Depending on whether a guide wire or a catheter is to be manufactured with the apparatus according to the invention, specific geometries for the spindle sleeve and the cannula device may be required corresponding to the number of the rod-shaped bodies.

Preferably, each rod-shaped body may have its own cannula provided in the desired geometrical arrangement. It is also possible, however, to supply two or more rod-shaped bodies through one cannula, for example. Furthermore, the cannulas can also be realized with a polygonal or oval cross-section.

When manufacturing catheters, a centrally arranged cannula serves for supplying compressed air for forming a catheter lumen.

The cannula devices for manufacturing guide wires and catheters may differ from each other for instance in that an apparatus for manufacturing catheters does not comprise a centering disc. By way of example, the apparatus for manufacturing guide wires which is shown in the first exemplary embodiment may be converted into an apparatus for manufacturing catheters through eliminating a fixing portion and the centering device. Thus, it is possible with the apparatus of the invention and also very advantageous that guide wires and catheters can be extruded with a single apparatus.

In the manufacture of guide wires, the discharge ends of the cannulas will terminate preferably in flush manner, i.e. they end in the manufacturing direction 2 at the same position.

When manufacturing catheters, provision is made that the external or peripheral cannulas terminate, as seen in the manufacturing direction, preferably further toward the rear with respect to the central cannula for supplying air. Regarding a catheter, the cannula set comprises the central cannula through which the supporting air pressure required for extruding the hose is built up. Further, the distance between the tip of the spindle sleeve and the discharge nozzle is of particular importance with the production of catheters in terms of the production results.

In the following, the functions of the individual components of the apparatus according to the invention will be generally described with reference to the further exemplary embodiments. These also apply if only one or more of the components which have been described above is/are present in the modified exemplary embodiments.

The supply disc between the material loom and the extruder serves for focusing the rod-shaped bodies and attenuating vibrations so that the rod-shaped bodies are exactly guided. This enhances the product quality.

If there is a large distance between the extrusion device and the material loom, the supply disc may be omitted. In this case, the material loom merely has to be provided with braked coils so that the rod-shaped bodies are under sufficient tension and do not oscillate too much. Using the supply disc allows to substantially reduce the length of the extrusion device without affecting the quality of the manufactured product.

For every rod-shaped body in the product geometry, the cannula set comprises an individual cannula, so that it is ensured that the rod-shaped bodies have the correct geometrical arrangement at the end in the manufacturing direction in the area in front of the spindle sleeve.

The apparatus according to the invention is suited for the very small dimensions of medical instruments, in particular catheters and guide wires. If the individual rod-shaped bodies were just be inserted into the spindle sleeve and sheathed with polymer, it would not be possible to achieve a useful geometric arrangement of the individual rod-shaped bodies in the medical instruments. This is due, on the one hand, to the irregular distribution of the rod-shaped bodies and the excessively high or excessively small distances of the individual rod-shaped bodies relative to one another or between the peripheral rod-shaped bodies and a central rod-shaped body. On the other hand, the peripheral rod-shaped bodies would not be uniformly covered with the polymer. It might even happen that the peripheral rod-shaped bodies project radially from the medical instrument or are exposed, and the medical instrument would be unusable.

For the purpose of achieving the geometrically correct arrangement, it is necessary to exactly guide the individual rod-shaped bodies in the correct geometry from the point of threading them in the spindle sleeve until the point of entering the extrusion space and further up to the point of leaving the outlet nozzle.

To this end, the cannula device is of vital importance. The cannula device has a number of cannulas, which are arranged in a cannula tube, which is exactly the same as the number of rod-shaped bodies provided in the desired geometry. The individual cannulas are firmly connected to one another, e.g. are soldered, and are provided with a screw thread (the adjustment device) which will then be screwed in the extrusion device in such a manner that the through-hole of the spindle sleeve is closed and the tip of the cannula device projects out of the spindle sleeve in the manufacturing direction.

The rod-shaped bodies are unwound from the material loom from braked coils, which are needed for generating the required tension in the rod-shaped bodies, are optionally passed through the supply disc and then threaded in the cannulas.

A further alignment disc may be provided in which the supply ends of the cannulas are fixed in such a manner that they are bent radially outward. This allows to thread the rod-shaped bodies in the cannulas without any rubbing, abrasion and bending load. In this way, supplying the rod-shaped bodies is facilitated. During starting up the apparatus, brass wires can be used instead of the rod-shaped bodies. The additional alignment disc facilitates the change from the brass wires to the rod-shaped bodies, in particular the process of inserting said bodies.

At some point at the end in the manufacturing direction, it might be necessary to again join the rod-shaped bodies, which are sheathed with polymer after having left the cannula set, by means of a centering device in order to stabilize the geometry. Due to the fact that the rod-shaped bodies will be compressed, the mutual distances of the individual rod-shaped bodies are reduced.

As seen in the manufacturing direction, the centering disc is arranged in the area upstream of the discharge nozzle. The cartwheel-like centering disc has a central centering hole through which a stabilization of the arrangement of the rod-shaped bodies in the desired geometry is achieved. Thereafter, the external dimension is formed by the nozzle diameter of the discharge nozzle.

For the purpose of successfully producing precisely the provided geometry, it might be the case that the mere presence of the components which have been described above is inadequate. It is also their exact positioning and their spacing with respect to one another and in coordination with the medical instrument to be produced which is of vital importance.

A cannula device for a guide wire geometry comprising one central and six peripheral rod-shaped bodies requires a central cannula and six peripheral cannulas radially spaced from it. The diameter of the cannulas has to be adapted in each case to the diameter of the rod-shaped bodies, so that the rod-shaped bodies can be passed through almost without any friction, yet preventing the polymer from entering the cannulas at the discharge end of the cannulas and clogging one or all cannulas.

It is preferred that rod-shaped bodies having a diameter of 0.24 mm are provided with cannula internal diameters of 0.3 mm or 0.4 mm, and rod-shaped bodies having a thickness of 0.17 mm will be provided with cannulas having a diameter of 0.3 mm. Smaller dimensions for the cannula diameter are absolutely conceivable. In order to make sure that the rod-shaped bodies—in the areas where the diameter is in its upper tolerance range—do not get stuck in the cannulas, the cannulas should have a diameter which is larger than that of the rod-shaped body, preferably by 10% to 80% and particularly preferred by 15% to 30%. As explained above, these tolerance values depend on the tolerance values of the rod-shaped bodies.

In case of guide wires, all the cannulas have approximately the same length at the discharge end. If there is no central rod-shaped body, the central cannula may be implemented as a solid rod which has no special functional relevance, but is provided only for establishing the geometrical arrangement of the cannulas.

In case of catheters, a central cannula of sufficient thickness is required to deliver the supporting air pressure with compressed air which is required for the extrusion of the hose. A central cannula of this type is connected to a compressed air supply device. Provision can be made here to arrange e.g. three cannulas in firmly soldered fashion, if three rod-shaped bodies are to be embedded in the catheter wall. Here, the cannulas through which the rod-shaped bodies are supplied can be realized so as to be shorter in the manufacturing direction, for instance can be arranged 4 to 12 mm, in particular 6 to 10 mm, behind the central cannulas.

With the extrusion of guide wires, the cannula set is arranged in such a manner that the discharge ends are spaced from the centering disc by approximately 3 to 5 mm. When extruding catheters, it is advantageous to position the central cannula as far as into the nozzle tip, because a better and more uniform catheter quality is achieved. It is also conceivable to do away with the centering disc during a catheter extrusion.

For the purpose of optimizing the production process in terms of economic efficiency, it may also be provided in a further configuration of the apparatus according to the invention to produce several guide wires or catheters in parallel with a single apparatus for extruding. It is conceivable, for example, to produce two or three strands in parallel. In doing so, two or three cannula devices will be arranged in the extrusion device, and two or three centering openings will then be present in the centering disc, so that two or three extrusion strands are pulled out side by side from a corresponding nozzle wall comprising two or three discharge nozzles and can be drawn through the water bath and cooled independently of one another side by side.

With an unchanged production volume, this process allows a correspondingly lower extrusion speed on each extrusion machine, resulting in a better product quality. Having the same extrusion speed, the production volume can be correspondingly increased. Accordingly, a correspondingly modified discharge nozzle head is required which has a somewhat more complex construction.

With alternative exemplary embodiments of the apparatus according to the invention, the supply device and/or the alignment device and/or the centering device may possibly be omitted.

The description of the apparatus according to the invention deals with a prototype. The final version may also be implemented without a discharge valve; in this case, the extrusion space would be similar to known extrusion machines (FIG. 18). This means that the extrusion space will have an annular cross-section and its height will show only a small distance to the spindle sleeve in order to exploit the molten mass in an efficient manner.

Especially with a medical instrument comprising only one rod-shaped body, the centering device may be omitted.

The apparatus according to the invention may also be used for manufacturing non-medical rod-shaped components such as hoses, for instance.

List of reference numerals

| | |
|---|---|
| 1 | apparatus |
| 2 | manufacturing direction |
| 3 | device for supplying rod-shaped bodies |
| 4 | coils |
| 5 | extrusion device |
| 6 | housing |
| 7 | side wall |
| 8 | discharge nozzle |
| 9 | supply wall |
| 10 | threaded rod |
| 11 | supply device |
| 12 | guiding hole |
| 13 | alignment device |
| 14 | alignment hole |
| 15 | cannula |
| 16 | cannula device |
| 17 | adjustment device |
| 18 | external thread |
| 19 | through-hole |
| 20 | supply end |
| 21 | discharge end |
| 22 | spindle sleeve |
| 23 | base wall |
| 24 | jacket wall |
| 25 | extrusion space |
| 26 | polymer supply device |
| 27 | through-hole |
| 28 | centering device |
| 29 | centering hole |
| 30 | melt opening |
| 31 | cooling device |
| 32 | exhaust device |
| 33 | roller device |
| 34 | supply portion |
| 35 | fixing portion |
| 36 | fixing portion |
| 37 | extrusion portion |
| 38 | circular segment |
| 39 | set screw |
| 40 | melt channel |
| 41 | cannula receptacle |
| 42 | heating device |
| 43 | melt pump |
| 44 | throttle |
| 45 | guide wire |
| 46 | fastening disc |
| 50 | rod-shaped bodies |
| 60 | polymer sheathing |
| 70 | catheter |
| 80 | catheter lumen |

1. An apparatus for extruding a medical instrument which can be inserted into a human or animal body, comprising
a device for supplying rod-shaped bodies,
an extrusion device comprising a housing, said housing having a surrounding side wall which, at the frontward end as seen in the manufacturing direction, is provided with a nozzle wall comprising a discharge nozzle and, at the rearward end opposite the manufacturing direction, is provided with a spindle sleeve, the space in the housing between the spindle sleeve, the side wall and the discharge nozzle delimiting an extrusion space and the housing being provided with a polymer supply device in the area of the extrusion space, and
a cannula device which extends in the manufacturing direction and is designed to insert at least one rod-shaped body from the device for supplying rod-shaped bodies into the extrusion space, which comprises at least one tubular cannula having a rearward supply end as seen in the manufacturing direction and a frontward discharge end as seen in the manufacturing direction, the cannula device being arranged approximately in straight alignment with respect to the discharge nozzle such that its discharge end situated in the manufacturing direction terminates at a distance from the discharge nozzle in the extrusion space.

2. The apparatus according to claim 1, for extruding a medical instrument which can be inserted into a human or animal body, comprising
a device for supplying rod-shaped bodies,
an extrusion device comprising a housing, said housing having a surrounding side wall which, at the frontward end as seen in the manufacturing direction, is provided with a nozzle wall comprising a discharge nozzle and, at the rearward end opposite the manufacturing direction, is provided with a spindle sleeve, the space in the housing between the spindle sleeve, the side wall and the discharge nozzle delimiting an extrusion space and the housing being provided with a polymer supply device in the area of the extrusion space, and
a cannula device which extends in the manufacturing direction and is designed to insert at least one rod-shaped body from the device for supplying rod-shaped bodies into the extrusion space, which comprises at least two tubular cannulas having a rearward supply end as seen in the manufacturing direction and a frontward discharge end as seen in the manufacturing direction, the cannula device being arranged approximately in straight alignment with respect to the discharge nozzle such that its discharge end situated in the manufacturing direction terminates at a distance from the discharge nozzle.

3. The apparatus according to claim 1, wherein
a supply device is arranged in the area between the device for supplying rod-shaped bodies and the supply end of the cannula device and is provided with a number of guiding holes, for guiding a rod-shaped body, which is equal to the number of rod-shaped bodies to be supplied.

4. The apparatus according to claim 1, wherein
a centering device is arranged in the area between the discharge end of the spindle sleeve and the discharge nozzle and comprises at least one centering hole which is preferably formed so as to taper in the manufacturing direction, to supply a rod-shaped body to the discharge nozzle in a predetermined position.

5. The apparatus according to claim 1, wherein
the arrangement of a melt channel of the discharge nozzle can be adjusted transverse to the manufacturing direction.

6. The apparatus according to claim 1, wherein
an alignment device is arranged in the area between the supply disc and the supply end of the cannula device and is provided with a number of alignment holes, for aligning the cannulas of the cannula device, which is equal to the number of cannulas.

7. The apparatus according to claim 1, wherein
a cooling device is arranged in the manufacturing direction downstream of the discharge nozzle.

8. The apparatus according to claim 1, wherein
a roller device designed to keep a medical instrument under tension is arranged in the manufacturing direction downstream of the cooling device.

9. The apparatus according to claim 1, wherein
a withdrawal device designed to withdraw a medical instrument from the apparatus is arranged in the manufacturing direction downstream of the roller device.

10. The apparatus according to claim 1, wherein
the cannula device comprises an adjustment device by means of which the distance in the manufacturing direction between a discharge end of a cannula and the centering device and/or discharge nozzle can be adjusted.

11. The apparatus according to claim 1, wherein
the cannula device comprises a central cannula for supplying air and a plurality of cannulas surrounding the central cannula and intended for supplying rod-shaped bodies.

12. A method for extruding a medical instrument which can be inserted into a human or animal body, in which
a rod-shaped body is supplied in a manufacturing direction by means of a device for supplying rod-shaped bodies to an extrusion device and is covered with a polymer in the extrusion device and
receives its final shape while leaving a discharge nozzle of the extrusion device in the manufacturing direction, wherein
the rod-shaped body is guided in the extrusion space and aligned by means of a tubular cannula of a cannula device of the extrusion device into the area in front of the discharge nozzle as seen in the manufacturing direction.

13. The method according to claim 12, wherein
the rod-shaped body, in the area between the device for supplying rod-shaped bodies and the supply end of the cannula device, is passed through a guiding hole of a supply device in order to stabilize/guide the rod-shaped body, and/or
the rod-shaped body, in the area between the discharge end of the cannula device and the discharge nozzle, is passed through a centering hole of a centering device in order to supply the rod-shaped body to the discharge nozzle in a predetermined position.

14. The method according to claim 13, wherein
during supplying a plurality of rod-shaped bodies, the interspaces between the rod-shaped bodies are coated with polymer, whereby they are embedded in the polymer.

15. The method according to claim 12, wherein
the final geometry of the medical instrument is determined by the nozzle diameter of the discharge nozzle while leaving the discharge nozzle, and/or
an alignment device comprising at least one alignment hole for aligning a cannula of the cannula device is arranged in the area between the supply disc and the supply end of the cannula device.
